(19) United States
(12) Patent Application Publication
(10) Pub. No.: US 2008/0027419 A1
(43) Pub. Date: Jan. 31, 2008

(54) Cryoprobe with Integral Agent Delivery Device

(75) Inventors: Kory P. Hamel, Bloomington, MN (US); David W. Vancelette, San Diego, CA (US); Douglas A. Devens JR., Higbland Park, IL (US)

Correspondence Address: AMS RESEARCH CORPORATION, 10700 BREN ROAD WEST, MINNETONKA, MN, 55343, US

(73) Assignee: AMS RESEARCH CORPORATION, Minnetonka, MN (US)

(21) Appl. No.: 11/828,132

(22) Filed: Jul. 25, 2007

Related U.S. Application Data
(60) Provisional application No. 60/820,293, filed on Jul. 25, 2006.

(51) Int. Cl. A61B 18/02

(52) U.S. Cl. 606/20

(57) ABSTRACT

A probe for use in a cryosurgical system has an integral device for delivering a treatment agent. A representative delivery device can take on a variety of forms, including a lumen located in an outer retractable sheath surrounding the cryoprobe tip, a channel attached to the side of the cryoprobe tip, a channel inside the cryoprobe tip, or a capsule located at the distal end of the cryoprobe tip. The delivery device is sealed to prevent air from being introduced into the system and is provided with a puncture member to break the seal and release the treatment agent as desired by a medical professional.

### PRIORITY CLAIM

The present application claims priority to U.S. Provisional Application No. 60/820,293, filed Jul. 25, 2006 and entitled “CRYOPROBE WITH INTEGRAL AGENT DELIVERY DEVICE, which is herein incorporated by reference in its entirety.

### FIELD OF THE INVENTION

The present disclosure relates to cryosurgical probes for use in the treatment of cancerous tumors or lesions, and more particularly to a cryoprobe having an integral device for delivery of a cryoprotective or cryodestructive agent.

### BACKGROUND OF THE INVENTION

Cryosurgical probes are used to treat a variety of diseases. Cryosurgical probes quickly freeze diseased body tissue, causing the tissue to die after which it will be absorbed by the body, expelled by the body, sloughed off or replaced by scar tissue. Cryothermal treatment can be used to treat prostate cancer and benign prostate disease. Cryosurgery also has gynecological applications. In addition, cryosurgery may be used for the treatment of a number of other diseases and conditions including breast cancer, liver cancer, glaucoma and other eye diseases.

A variety of cryosurgical instruments variously referred to as cryoprobes, cryosurgical probes, cryosurgical ablation devices, cryostats and cryocoolers have been used for cryosurgery. These devices typically use the principle of Joule-Thomson expansion to generate cooling. They take advantage of the fact that most fluids, when rapidly expanded, become extremely cold. In these devices, a high pressure gas mixture is expanded through a nozzle inside a small cylindrical shaft or sheath typically made of steel. The Joule-Thomson expansion cools the steel sheath to a cold temperature very rapidly. The cryosurgical probes then form ice balls which freeze diseased tissue. A properly performed cryosurgical procedure allows cryoablation of the diseased tissue without undue destruction of surrounding healthy tissue.

However, there is still a risk that during cryosurgery, healthy tissues, nerves, or blood vessels will be accidentally frozen. Such accidental freezing can cause additional complications, such as incontinence or impotence. In order to help avoid such accidental freezing, certain chemical compounds, known as cryoprotective or cryophylactic agents, can be used on the surrounding healthy tissues to help prevent them from becoming frozen. In addition, certain other agents, known as cryodestructive agents, can be used to enhance freezing of the target tissue.

### SUMMARY OF THE INVENTION

The present disclosure is directed to a probe for use in a cryosurgical system that has an integral delivery device for delivering a treatment element at a desired stage of a cryosurgical procedure. The integral delivery device can take on a variety of forms, including a sealed lumen located in an outer retractable sheath surrounding a cryoprobe tip, a sealed channel attached to the side of the cryoprobe tip, a sealed channel inside the cryoprobe tip, or a sealed capsule located at a distal treatment end of the cryoprobe tip. Generally, the delivery device comes sealed to prevent air from being introduced into the system and to allow for selective delivery and application of the treatment element and a treatment site. Based upon the treatment to be performed, the treatment element can comprise a treatment agent, a treatment sensor, a treatment device and various combinations thereof. In some representative embodiments, the integral delivery device can provide for the delivery of a cryodestructive agent or a cryoprotective agent to the treatment site. Depending upon the treatment element, the probe can further comprise a puncture member allowing an operator to selectively puncture the integral delivery device so as to break a seal prior to delivery of the treatment element.

In one aspect of the present disclosure, a cryosurgical probe can comprise a probe tip and a sealed delivery device. The sealed delivery device is generally adapted to contain a treatment element, a treatment sensor, a treatment device and combinations thereof. In some representative embodiments, the sealed delivery device can allow for the selective delivery and application of a cryoprotective or cryodestructive agent onto a surrounding or target region of a treatment site at the beginning of an operation. The sealed delivery device is integrally attached to the cryoprobe tip such that delivery of the treatment element as well as use of the cryosurgical probe to perform cryosurgery is accomplished with the same cryosurgical probe. The cryosurgical probe can comprise a puncture member allowing a medical professional to selectively pierce or otherwise puncture the sealed delivery device to allow for application, approximation, injection or insertion of the treatment element at the treatment site.

In another aspect of the present disclosure, a method for cryosurgical treatment of tissue can comprise providing a cryosurgical probe having a probe tip with an integral sealed delivery element. The cryosurgical probe can be positioned such that the probe tip and integral sealed delivery element are positioned proximate a treatment site. Once the integral sealed delivery element is suitably positioned, the integral sealed delivery element can be punctured or otherwise pierced such that a treatment element contained within the integral sealed delivery element can be applied, approximated, injected or otherwise inserted at the treatment site. Depending upon the treatment to be performed, the integral sealed delivery element can be pierced prior to, during or after a cryosurgical procedure performed with the cryosurgical probe. In one representative embodiment, a cryoprotective or cryodestructive agent can be applied to the treatment site or surrounding tissue prior to the cryosurgical procedure.

In yet another aspect of the present disclosure, a cryosurgical treatment system can comprise a closed loop cryosurgical system including one or more cyroprobes having a sealed delivery channel containing a treatment element. The closed loop cryosurgical system allows for selective freezing of target regions of tissue with the cryoprobe while simultaneously delivering the treatment element to the target region. At an appropriate time, either prior to, during or after the cryosurgical procedure is performed, the sealed delivery channel can be punctured or otherwise pierced such that the treatment element can be applied, approximated, injected or otherwise inserted at the target region. In one representative embodiment, a cryoprotective or cryodestructive agent can be applied to either the target region or surrounding tissue prior to the cryosurgical procedure.

The above summary of the various representative embodiments of the invention is not intended to describe each illustrated embodiment or every implementation of the invention. Rather, the embodiments are chosen and described so that others skilled in the art may appreciate and understand the principles and practices of the invention. The figures in the detailed description that follows more particularly exemplify these embodiments.

### BRIEF DESCRIPTION OF THE FIGURES

These as well as other objects and advantages of this invention, will be more completely understood and appreciated by referring to the following more detailed description of the presently preferred exemplary embodiments of the invention in conjunction with the accompanying drawings of which:

FIG. 1 is a front view of cryoprobe according to an embodiment of the present invention.

FIG. 2 is a section view of a cryoprobe according to an embodiment of the present invention.

FIG. 3 is a front view of a cryoprobe according to an embodiment of the present invention.

FIG. 4 is a side view of a cryoprobe according to an embodiment of the present invention.

FIG. 5 is a front view of a cryoprobe according to an embodiment of the present invention.

FIG. 6 is a partial section view of a cryoprobe according to an embodiment of the present invention.

FIG. 7 is a partial section view of a cryoprobe according to an embodiment of the present invention.

FIG. 8 is a section view of a cryoprobe according to an embodiment of the present invention.

FIG. 9 is a section view of a cryoprobe according to an embodiment of the present invention.

FIG. 10 is a section view of a cryoprobe according to an embodiment of the present invention.

### DETAILED DESCRIPTION OF THE PREFERRED EMBODIMENTS

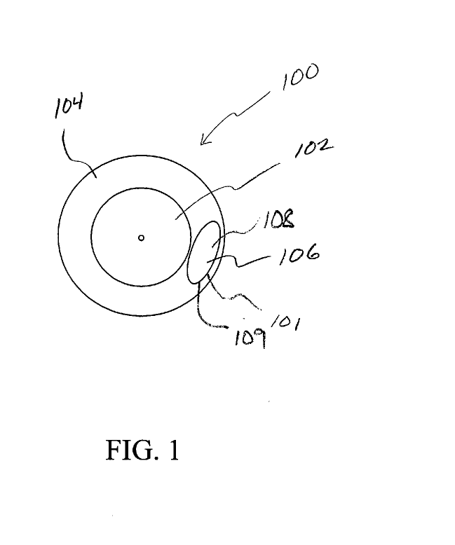

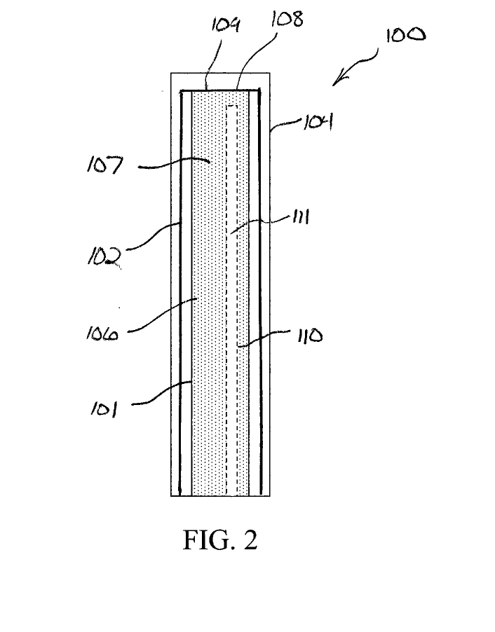

Referring to FIGS. 1 and 2, there can be seen a representative cryoprobe 100 comprising an integral agent delivery device 101 according to an embodiment of the present invention. Cryoprobe 100 generally includes a sheath 104 surrounding a cryoprobe tip 102 that has an integral delivery lumen 106 there through. Sheath 104 is preferably thin-walled and can comprise a tube made of a suitable, medical grade plastic. Integral delivery device 106 generally includes a sealed end 108 such that a treatment agent 107 is isolated therein. Sealed end 108 includes a seal 109 to minimize air provide for selective introduction of treatment agent 107. Intergal delivery device further comprises a puncture member 110 such as, for example, a wire 111 or other similar puncturing or piercing means allowing a medical professional to selectively release or otherwise apply the treatment agent 107 as desired. Integral delivery device 106 can further comprise an external biasing element such as, for example, a syringe plunger to optionally pressurize the treatment agent 107 for injection, or alternatively, apply negative pressure to remove the treatment agent 107 following a cryosurgical procedure. Although depicted as utilizing a single integral delivery lumen 106 for delivery of treatment agent 107, cryoprobe 100 may comprise a plurality of integral delivery lumens where desired based upon the treatment to be performed and/or the location of the treatment site.

In one representative embodiment, treatment agent 107 can comprise a cryodestructive agent. Utilizing cryoprobe 100, the selected cryodestructive agent can be injected into or otherwise applied to target regions of tissue prior to the cryosurgical procedure so as to enhance the freezing process at the target region. Representative examples of cryodestructive agents that are contemplated for use with the cryoprobe 100 can include, for example, water, ethanol, glycol, TNF-α, gold nanoparticles with TNF-α, other metallic nanoparticles (such as aluminum or Fe₃O₄), arsenic trioxide, DMXAA, pH lowering agents, chemotherapy agents such as Taxotere, Cisplatin, 5-FU, Peplomycin, or Adriamycin, Erk Inhibitors, Mitochondrial specific agents such as Cyclosporine A, Antimycin, or PK 11195, NaCl, AFP, solutions with low latent heat, solution with high thermal conductivity, or some combination of these. By integrally forming cryoprobe 100 so as to include a cryodestructive agent within integral delivery device 106, desired cryoinjury of the selected tissue can be enhanced in a simple and efficient manner with little alteration to the typical cryosurgical procedure.

In another representative embodiment, treatment agent 107 can comprise a cryoprotective agent such that cryoprobe 100 can be utilized to protect tissue surrounding a target region from accidental freezing. The cryoprotective agent can be injected into or otherwise applied to the surrounding tissue prior to conducting a cryosurgical treatment with the cryoprobe 100. Representative examples of suitable cryoprotective agents that can be used with cryoprobes of the present disclosure include glycerol, propylene, glycol, DMSA, DMSO, AFP, glucose, VM3, VEG, or some combinations of these. By integrally forming cryoprobe 100 so as to include a cryoprotective agent within integral delivery device 106, protection of selected tissue from exposure to freeze-induced cryoinjury is accomplished in a simple and efficient manner without significantly altering the typical cryosurgical procedure.

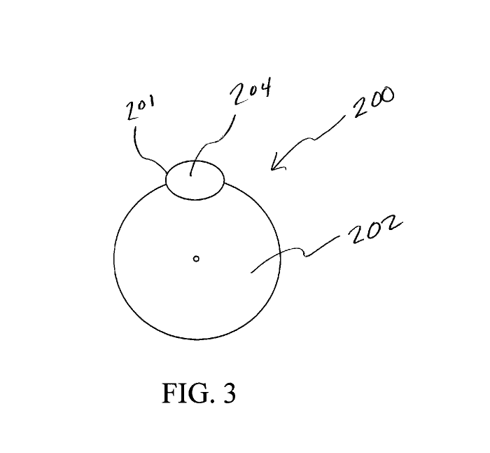

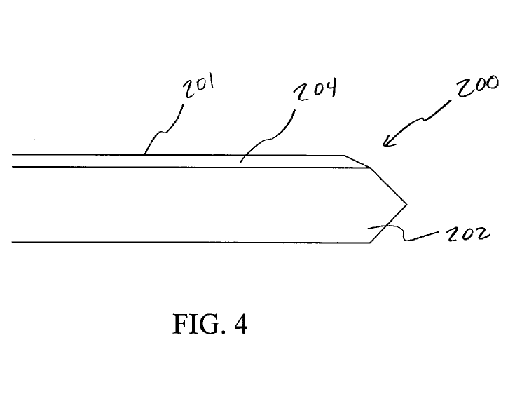

Referring now to FIGS. 3 and 4, there can be seen a cryoprobe 200 with an integral agent delivery device 201 according to another embodiment of the present disclosure. Cryoprobe 200 generally includes a delivery channel 204 adjacent a cryoprobe tip 202 for delivery of the treatment agent 107. In one embodiment, cryoprobe 200 is formed such that delivery channel 204 is integrally formed with the cyroprobe 200. Alternatively, cryoprobe 200 can comprise an assembly wherein delivery channel 204 is separately attached or coupled to an existing cryoprobe tip 202. As with cryoprobe 100, delivery channel 204 of cryoprobe 200 is sealed and contains treatment agent 107 such that puncture member 110 can be utilized to puncture or otherwise pierce the delivery channel 204 so as to release or otherwise apply the treatment agent 107 at the targeted region of tissue. Channel 204 can also optionally be used for subsequent removal of the treatment agent 107 following the cryosurgical procedure.

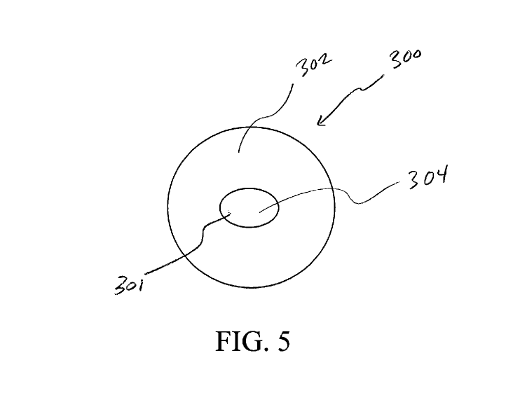

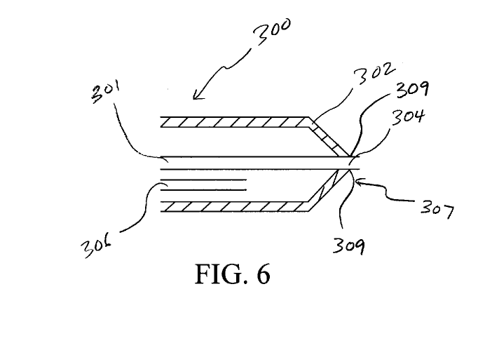

FIGS. 5, 6 and 7 depict a cryoprobe 300 with an integral agent delivery device 301 according to another representative embodiment of the present invention. Integral agent delivery device 301 generally comprises a delivery lumen 304 within a cryoprobe tip 302 located along side capillary tubing 306 or other suitable expansion element used to expand high pressure refrigerant in the cryoprobe 300. As illustrated in FIG. 6, the delivery lumen 304 is in fluid communication with a distal end 307 of the cryoprobe tip 302. At the distal end 307, the cryoprobe tip 302 is hermetically sealed with a tip seal 309 positioned over delivery lumen 304 so to prevent the introduction of air into the cryoprobe 300 and to retain treatment agent 107 within the delivery lumen 304. Hermetic sealing may be accomplished by any suitable means, such as, for example, laser or electron beam welding. In addition, as with the above described embodiments, delivery lumen 304 can be provided with a puncture member 110 to puncture or otherwise pierce tip seal 309 and allow for the selective delivery and/or introduction of the treatment agent 107 either prior to, during or following the cryosurgical procedure.

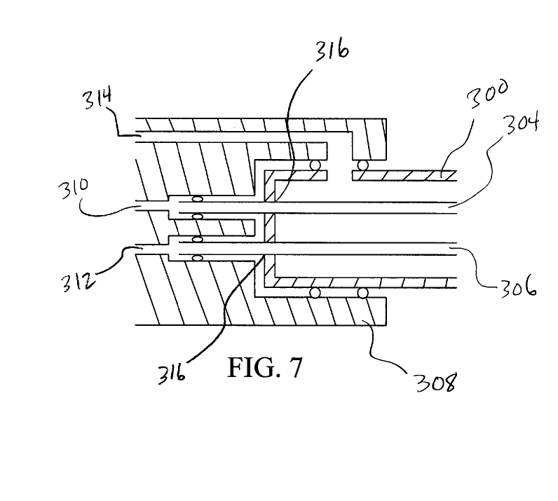

As illustrated in FIG. 7, cryoprobe 300 can be operably connected to a refrigerant delivery system via a probe connector 308. When treatment agent 107 comprises a cryoprotective or cryodestructive agent, the agent enters the delivery lumen 304 from the refrigerant delivery system through an access port 310 in probe connector 308. High pressure refrigerant, such as, for example, any of various appropriate gas mixtures that are known in the art, enters the capillary tubing 306 through an adjacent access port 312 and returns as low pressure refrigerant through a channel 314. Hermetic seals exist at a proximal seal 316 on cryoprobe 300 wherein the proximal seal 316 interfaces with delivery lumen 304 and capillary tubing 306 to prevent the introduction of air into the system.

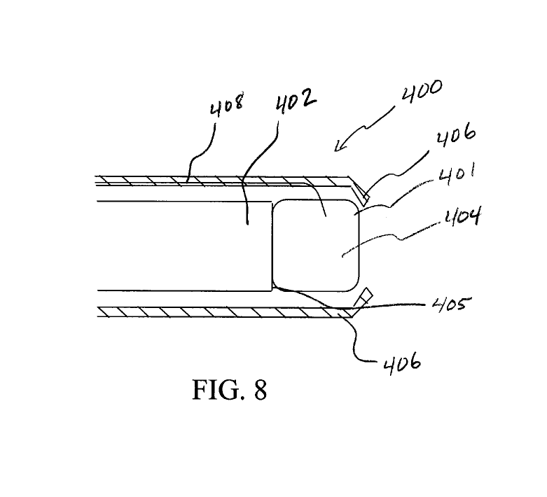

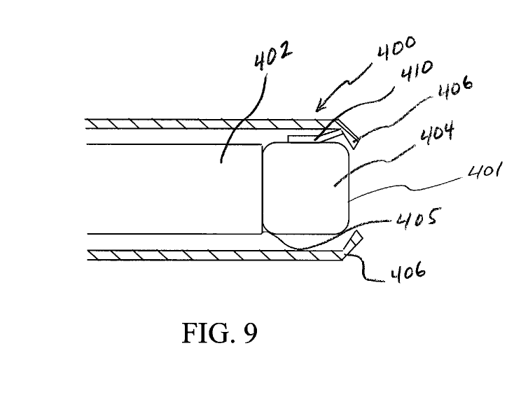

Referring now to FIGS. 8 and 9, there can be seen a cryoprobe 400 having an integral agent delivery device 401 according to a further embodiment of the present invention. Integral agent delivery device 401 generally includes a balloon-like capsule 404 within which treatment agent 107, such as, for example, a cryoprotective or cryodestructive agent is contained. Balloon-like capsule 404 is generally positioned at a distal end 405 of a cryoprobe tip 402. Capsule 404 can be enclosed within a protective sheath 406 surrounding the cryoprobe tip 402 so as to maintain and protect the integrity of capsule 404. To apply treatment agent 107, capsule 404 can be punctured or otherwise pierced either prior to or after sheath 406 is withdrawn from over capsule 404. As shown in FIG. 8, the sheath 406 may contain a puncture or piercing member such as, for example, wire 408 or other suitable puncturing or piercing means to rupture the capsule 404 as the sheath 406 is withdrawn. Alternatively, as shown in FIG. 9, a strip of material 410 may be adhered to both the sheath 406 and the capsule 404 such that that withdrawal of the sheath 406 causes the strip 410 to tear open the capsule 404 and release the treatment agent 107.

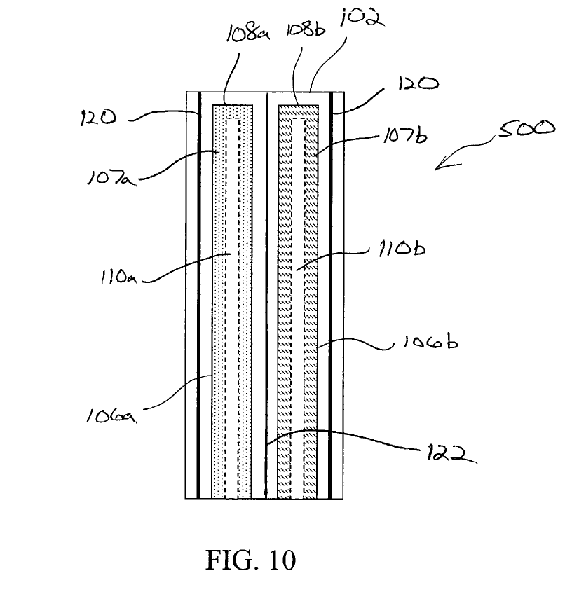

In addition to providing for the introduction of cryoprotective and cryodestructive agents, representative cryoprobes of the present disclosure can comprise various additional features to enhance the efficacy, ease of use and outcomes of a cryosurgical treatment while reducing the potential for cryoinjury. For example, thermosensors may be inserted into various regions of interest in order to monitor the temperature of areas targeted for cryoablation and areas that are not to be frozen and/or to be warmed. In addition, heating probes may be inserted into surrounding tissue or other nearby areas of the body for warming select regions in order to prevent them from freezing during cryosurgery. Such a system may incorporate multiple probes wherein some probes are used for heating and some probes are used for freezing. Further, additional treatments, such as chemotherapy, radiation treatment, or percutaneous ethanol injection therapy, may be combined with cryosurgery in order to enhance patient treatment. The various cryoprobes of the present disclosure including cryoprobes 100, 200, 300 and 400, used individually or in combination, can be utilized to enhance the efficacy and cryosurgical outcome of a cryosurgical treatment such as, for example, cryosurgical treatment of prostate cancer. For illustrative purposes, use of a cyroprobe 500 is described though it will be understood that cryoprobes 100,200, 300 and 400 can be similarly employed. Referring to FIG. 10, cryoprobe 500 generally resembles the look and construction of cryoprobe 100 with the further inclusion of a pair of integral delivery devices 106a, 106b. Generally, cryoprobe 500 and more specifically, the cryoprobe tip 102 can be positioned proximate cancerous tissue on the prostate using a suitable medical imaging technology. With cryoprobe tip 102 positioned thus, a medical professional can withdraw sheath 104 so as to expose sealed end 108a of integral delivery device 106a and sealed end 108b of integral delivery device 106b to the treatment site. Using puncture member 110a, a cryodestructive agent 107a can be injected or otherwise applied to the cancerous tissue so as to promote tissue freezing during the cryosurgical procedure. Using puncture member 11Ob, a cryoprotective agent 107b can be applied to surrounding healthy tissue to the protect the healthy tissue from freezing. Cryoprobe 100 can further delivery a variety of sensors or probes to assist with the cryosurgical treatment. For instance, cryoprobe 100 can include one or more thermosensors 120 allowing the medical professional to monitor the temperatures of both target and surrounding tissue during the cryosurgical procedure. Cryoprobe 500 can further include a heating probe 122 that can be inserted into the prostate or surrounding tissue to heat and protect healthy tissue from freezing during the cryosurgical procedure.

Through the use of the various cryoprobes as described above, the opportunity for improved cryosurgical outcomes in enhanced. Treatment enhancements include enhanced cryoinjury regions, faster treatment times, cryotreatment at higher (and therefore safer) temperatures, greater localization of cryodamage, and improved protection of healthy tissue regions not targeted for freezing.

The above described embodiments of cryoprobes can be used with various types of cryoablation systems. In one presently preferred embodiment, the described cryoprobes are used with a closed-loop cryosurgical system. One such suitable system is described in co-pending U.S. Provisional Patent Application Ser. No. 60/820,290, filed Jul. 25, 2006, entitled “CLOSED LOOP CRYOSURGICAL SYSTEM,” which is hereby incorporated in its entirety by reference.

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products.

What is claimed is:

1. A probe for use in a cryosurgical procedure comprising:
a tip conFIG.d to freeze selected tissue during a cryosurgical procedure;
an integral delivery device located proximate the tip, the integral delivery device retaining a treatment agent therein; and
a puncture member for releasing the treatment agent from the integral delivery device.

2. The probe of claim 1, wherein the integral delivery device comprises a delivery lumen extending to a sealed end on the tip and wherein the puncture member pierces a seal on the sealed end to release the treatment agent.

3. The probe of claim 2, wherein the delivery lumen is integrally formed with the tip.

4. The probe of claim 2, wherein the delivery lumen is adjacent a capillary tube and wherein the delivery lumen is fluidly connected to a first access port for receiving the treatment agent and the capillary tube is fluidly connected to a second access port for receiving a high pressure refrigerant.

5. The probe of claim 1, wherein the integral delivery device comprises a capsule member attached to the tip and wherein the puncture member ruptures the capsule member to release the treatment agent.

6. The probe of claim 5, wherein the puncture member comprises a sheath positioned over capsule member such that withdrawal of the sheath ruptures the capsule member.

7. The probe of claim 1, wherein the treatment agent comprises a cryodestructive agent selected from the group consisting of: water, ethanol, glycol, TNF-α, metallic nanoparticles, arsenic trioxide, DMXAA, a pH lowering agent, a chemotherapy agent, ERK inhibitors, a mitochondrial agent, sodium chloride, and AFP.

8. The probe of claim 1, wherein the treatment agent comprises a cryoprotective agent selected from the group consisting of: glycerol, propylene, glycol, DMSA, DMSO, AFP, glucose, VM3, and VEG.

9. The probe of claim 1, wherein the integral delivery device comprises a plurality of integral delivery devices for retaining a plurality of treatment agents therein.

10. A method of performing cryosurgery, comprising:
providing a cryoprobe having a cryoprobe tip and an integral delivery device, the integral delivery device sealably retaining a treatment agent;
positioning the cyropobe tip proximate a treatment site; and
releasing the treatment agent from the integral delivery device.

11. The method of claim 10, wherein releasing the treatment agent comprises puncturing a seal on the integral delivery device with a puncture member.

12. The method of claim 10, wherein releasing the treatment agent comprises rupturing the integral delivery device by withdrawing a sheath from over the integral delivery device.

13. The method of claim 10, further comprising:
enhancing the freezing of tissue at the treatment site by releasing a cryodestructive treatment agent from the integral delivery device.

14. The method of claim 10, further comprising:
performing a cryosurgical treatment at the treatment site.

15. The method of claim 14, wherein the cryosurgical treatment comprises a prostate cancer cryosurgical treatment.

16. The method of claim 14, further comprising:
removing the treatment agent from the treatment site with the integral delivery device.

17. The method of claim 10, wherein the integral delivery device comprises a first integral delivery device and a second integral delivery device and wherein releasing the treatment agent from the integral delivery device comprises releasing a first treatment agent from the first integral delivery device and releasing a second treatment agent from the second integral delivery device.
